(19) United States
(12) Patent Application Publication
(10) Pub. No.: US 2011/0092883 A1
(43) Pub. Date: Apr. 21, 2011

(54) TRANSDERMAL DRUG ADMINISTRATION APPARATUS AND TRANSDERMAL DRUG ADMINISTRATION UNIT

(76) Inventor: Kenichi UCHIYAMA, Chigasaki-shi (JP)

(21) Appl. No.: 12/904,476

(22) Filed: Oct. 14, 2010

(30) Foreign Application Priority Data
Oct. 16, 2009 (JP) 2009-239509

(51) Int. Cl. A61M 5/00; A61N 1/30

(52) U.S. Cl. 604/21; 604/272; 604/173; 604/182

(57) ABSTRACT

According to one embodiment, a transdermal drug administration apparatus includes a first member having a protrusion on a facing surface opposed to a skin and a guide hole in the protrusion and a second member having a needle portion located in the guide hole and movable in a first direction across a surface of the skin.

### CROSS-REFERENCE TO RELATED APPLICATIONS

This application is based upon and claims the benefit of priority from Japanese Patent Application No. 2009-239509, filed Oct. 16, 2009; the entire contents of which are incorporated herein by reference.

### FIELD

Embodiments described herein relate generally to a transdermal drug administration apparatus and a transdermal drug administration unit configured to administer a drug through the skin.

### BACKGROUND

In general, transdermal drug administration apparatuses that administer drugs through the skin have the problem of a low drug absorption rate, so that various measures have been taken to enhance the drug absorption rate. For example, there are chemical approaches that use absorption enhancers, such as ethanol, isopropyl myristate, etc., and physical approaches that use electrical energy, such as Iontophoresis and electroporation, to enhance the drug absorption rate. Further, a transdermal drug administration apparatus has been proposed in which microneedles for mechanically perforating the skin to facilitate drug infiltration are integrated with a reservoir that holds a drug. In one such administration apparatus, a plate-like member comprising a large number of microneedles and solution paths lies below a tank that holds the drug (e.g., WO2006/016647). Also provided is a transdermal drug administration apparatus in which microneedles are guided at right angles to the skin by means of a flexible seat that moves step by step, as disclosed in Jpn. PCT National Publication No. 2008-520369.

### BRIEF DESCRIPTION OF THE DRAWINGS

FIG. 1 is a diagram showing the internal structure of a transdermal drug administration apparatus according to a first embodiment;

FIG. 2A is a bottom perspective view showing an external configuration of the administration apparatus in a retracted state;

FIG. 2B is a bottom perspective view showing an external configuration of the administration apparatus in an advanced state;

FIG. 3A is a top perspective view showing an external configuration of the administration apparatus with its push button not depressed;

FIG. 3B is a top perspective view showing an external configuration of the administration apparatus with the push button depressed;

FIG. 4 is a perspective view showing a configuration of a needle portion of the administration apparatus;

FIG. 5A is a diagram showing a drug administration method using the transdermal drug administration apparatus;

FIG. 5B is a diagram showing a drug administration method using the transdermal drug administration apparatus;

FIG. 5C is a diagram showing a drug administration method using the transdermal drug administration apparatus;

FIG. 6A is a perspective view showing an external configuration of a transdermal drug administration unit according to a second embodiment with its push button not depressed;

FIG. 6B is a perspective view showing an external configuration of the administration unit of the second embodiment with the push button depressed;

FIG. 7A is a diagram showing the internal structure of a transdermal drug administration apparatus according to a third embodiment and a drug administration method;

FIG. 7B is a diagram showing the internal structure of a transdermal drug administration apparatus according to a third embodiment and a drug administration method;

FIG. 7C is a diagram showing the internal structure of a transdermal drug administration apparatus according to a third embodiment and a drug administration method;

FIG. 8A is a bottom perspective view showing an external configuration of the administration apparatus in a retracted state;

FIG. 8B is a bottom perspective view showing an external configuration of the administration apparatus in an advanced state;

FIG. 9 is a diagram showing the internal structure of a transdermal drug administration apparatus according to a fourth embodiment; and

FIG. 10 is a diagram showing the internal structure of a transdermal drug administration apparatus according to a fifth embodiment.

### DETAILED DESCRIPTION

In general, according to one embodiment, a transdermal drug administration apparatus comprises a first member comprising a protrusion on a facing surface opposed to a skin and a guide hole in the protrusion and a second member comprising a needle portion located in the guide hole and movable in a first direction across a surface of the skin.

A transdermal drug administration unit according to another embodiment comprises the transdermal drug administration apparatus and an attachment seat comprising an attachment portion mounted on the transdermal drug administration apparatus and capable of adhering to the skin in a region around the facing surface.

### First Embodiment

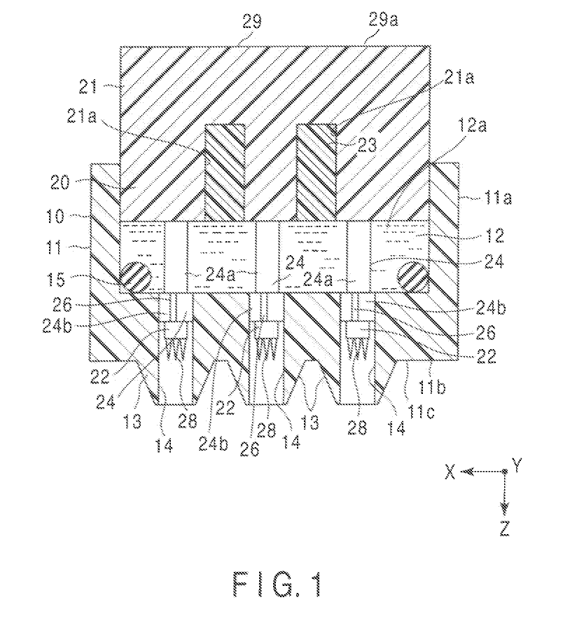

A transdermal drug administration apparatus 1 according to a first embodiment will now be described with reference to FIGS. 1 to 5C. In each of these schematic drawings, configurations are enlarged, reduced, or omitted as required for ease of explanation. In these drawings, arrows X, Y and Z individually indicate three orthogonal directions. In this case, a treatment is performed for an underlying skin 30, and an advancing direction is assumed to be downward (Z-direction) and a retreating direction to be upward.

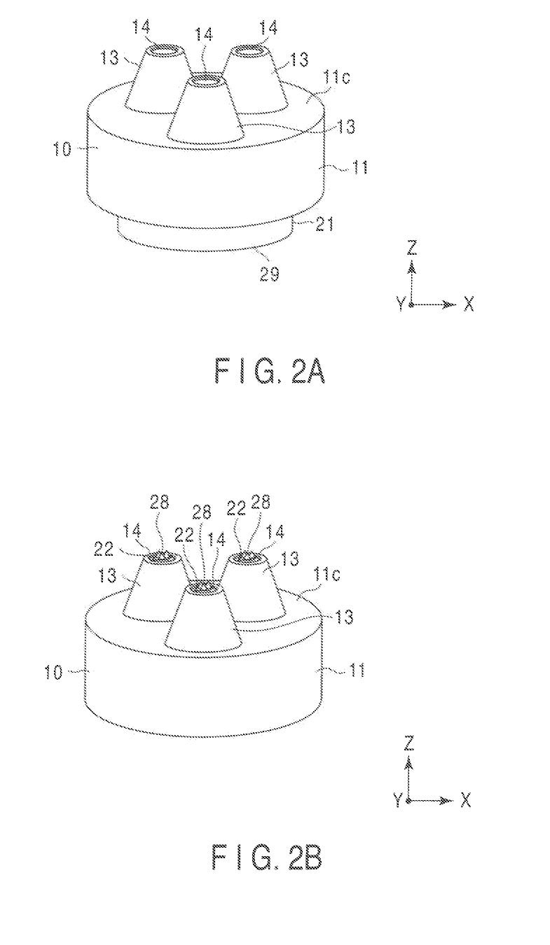

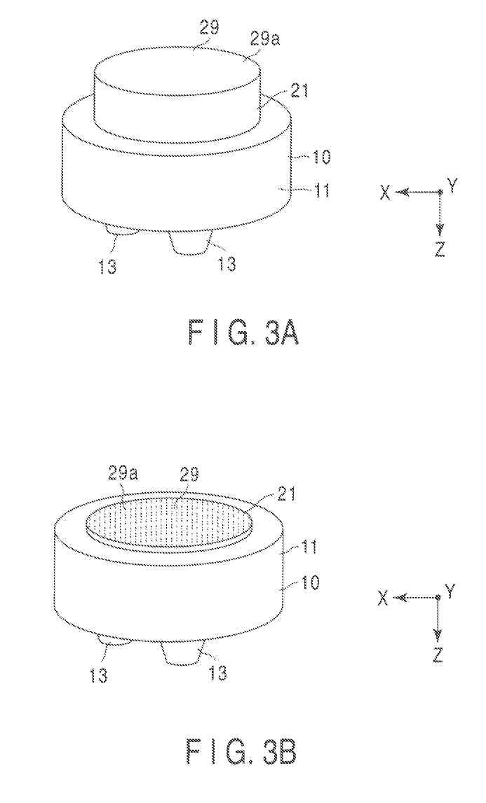

FIG. 1 is a diagram showing the internal structure of the transdermal drug administration apparatus 1 according to the first embodiment. FIGS. 2A and 2B, which are bottom perspective views showing an external configuration of the administration apparatus 1. FIGS. 2A and 2B show a retracted state and an advanced state, respectively. FIGS. 3A and 3B, which are top perspective views showing an external configuration. FIGS. 3A and 3B show states before and after button depression, respectively.

The transdermal drug administration apparatus 1 shown in FIGS. 1 to 3B is an apparatus for delivering a drug (medicine) 12a to the skin 30. The administration apparatus 1 comprises a base section (first member) 10 to be attached to the skin 30 and a slide section (second member) 20 slidably fitted in the base section 10. In the apparatus 1, the slide section 20 moves guided by the base section 10 pressed against the skin 30. As the slide section 20 moves in this manner, needle portions 22 of the slide section 20 move in a direction (Z-direction) perpendicular to the surface (XY-plane) of the skin 30, as a subject area to be pierced, thereby contacting or separating from the skin 30.

The base section 10 is formed of, for example, a plastic material. The base section 10 comprises a casing 11, which defines a storage recess 12 for accommodating a liquid drug 12a. The casing 11 comprises an annular peripheral wall portion 11a having its central axis extending in the Z-direction and a disk-like bottom portion 11b and defines the storage recess 12, a circular space, therein. The top side of the storage recess 12 is open, and the slide section 20 is fitted in the base section 10 so as to close the top opening. The liquid drug 12a is held in the recess 12.

Three protrusions 13 protrude in the Z-direction (first direction) toward the skin 30 from the lower surface of the bottom portion 11b, which serves as a facing surface 11c that faces the skin 30. The protrusions 13 bulge in, for example, hemispheres or frustums. The protrusions 13 bite into and spread the skin 30 when the facing surface 11c is pressed against the skin.

A plurality of guide holes 14 extending in the Z-direction are individually formed in the bottom portion 11b of the casing 11. The guide holes 14 penetrate the bottom portion 11b in the Z-direction at the respective center tops of the protrusions 13 and internally connect the storage recess 12 above them and the side of the facing surface 11c of the casing 11. The needle portions 22 of the slide section 20 are inserted into the guide holes 14, individually. The needle portions 22 are guided in the Z-direction by the respective inside walls of the guide holes 14.

An elastically deformable O-ring 15 (elastic member) is fitted between a moving part 21 of the slide section 20 and the casing 11 of the base section 10. The O-ring 15 is located along, for example, a corner portion between the peripheral wall portion 11a and bottom portion 11b of the casing 11. If a push button 29 is depressed, the O-ring 15 is compressed and elastically deformed between the base and slide sections 10 and 20. If the button 29 is released from the depression, the O-ring 15 pushes back, by its elastic restoring force, the slide section 20 away from the bottom portion 11b of the base section 10 or in a direction such that the slide section 20 recedes from the skin 30.

The slide section 20 comprises the moving part 21, which is fitted in the storage recess 12, and the three needle portions 22 extending in the Z-direction. The top side of the moving part 21 constitutes the push button (pushable portion) 29. The moving part 21, e.g., a plastic disk, closes the top opening of the storage recess 12 and is movable in the Z-direction, guided by the inside wall of the recess 12. The drug 12a in the storage recess 12 is pressurized as the moving part 21 moves. The three needle portions 22 are connected to the moving part 21.

Elastic member holes 21a are formed in the underside of the moving part 21, and elastically-compressed members (compressed elastic portions) 23 are embedded in the holes 21a, individually. The compressed members 23 are interposed between the drug 12a and moving part 21 so as to be exposed on the side of the storage recess 12. If the push button 29 is depressed so that the moving part 21 moves downward, the compressed members 23 are compressed and reduced in size between the drug 12a and moving part 21. After the button 29 is released from the depression, the compressed members 23 are enlarged by their restoring force as they push out the drug 12a in the Z-direction.

An indicator 29a is formed on the top surface of the push button 29. Pressure-sensitive ink in the form of microcapsules is printed on the indicator 29a. As shown in FIGS. 3A and 3B, the pressure of depression of the button 29 causes the ink on the indicator 29a to develop a color, thereby indicating that the skin 30 is punctured with microneedles 28a, 28b and 28c. This indication enables a user to visually check to see if the button is depressed. In FIG. 3B, the indicator 29a is hatched in violet or purple. However, the color produced by the indicator 29a is not limited to violet or purple and may be another.

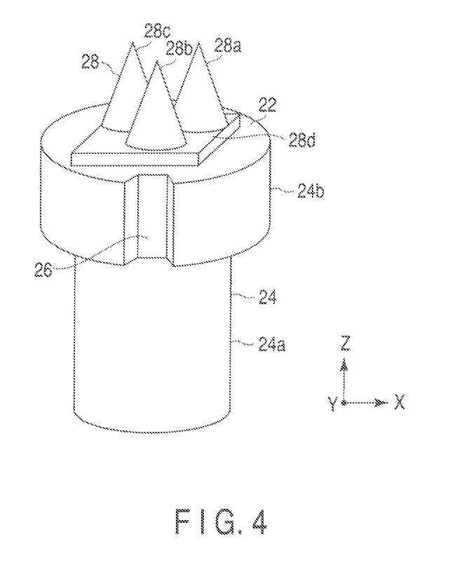

FIG. 4 is a perspective view showing one of the needle portions 22. As shown in FIGS. 1 and 4, the three needle portions 22 each comprise a shaft 24 and microneedle unit 28. The shaft 24 advances and retreats in the Z-direction in each corresponding guide hole 14. The microneedle unit 28 is provided on the distal end side of the shaft 24. The shaft 24 comprises, for example, a small-diameter portion 24a smaller in diameter than the guide hole 14 and a large-diameter portion 24b larger than the small-diameter portion 24a and guided by the guide hole 14. Within the guide hole 14, the shaft 24 moves in the Z-direction, guided by the inside wall of the guide hole, and advances or retreats so as to contact or separate from the skin 30.

The outer peripheral surface of the large-diameter portion 24b is provided with a guide groove 26, which is formed extending from one axial end of the portion 24b to the other. As the moving part 21 moves, the drug 12a held in the storage recess 12 is delivered from the facing surface 11c to the skin 30 outside the casing 11 through a space around the small-diameter portion 24a in each guide hole 14 and the guide groove 26 of each large-diameter portion 24b. Thus, the guide hole 14 functions as a guide for its corresponding needle portion 22 and also as a part of a communication path through which the drug 12a is guided.

The microneedle units 28 are formed of, for example, silicon. Each microneedle unit 28 comprises one or more sharp microneedles 28a to 28c on a sheet-like substrate 28d. The microneedles 28a to 28c are formed by, for example, a microarray technology, in which a microneedle array is created on the basis of a microprocessing technology. When the microneedles 28a to 28c pierce the skin 30 as they advance and project from the facing surface 11c of the casing 11, the barrier of the skin 30 is physically broken and perforated.

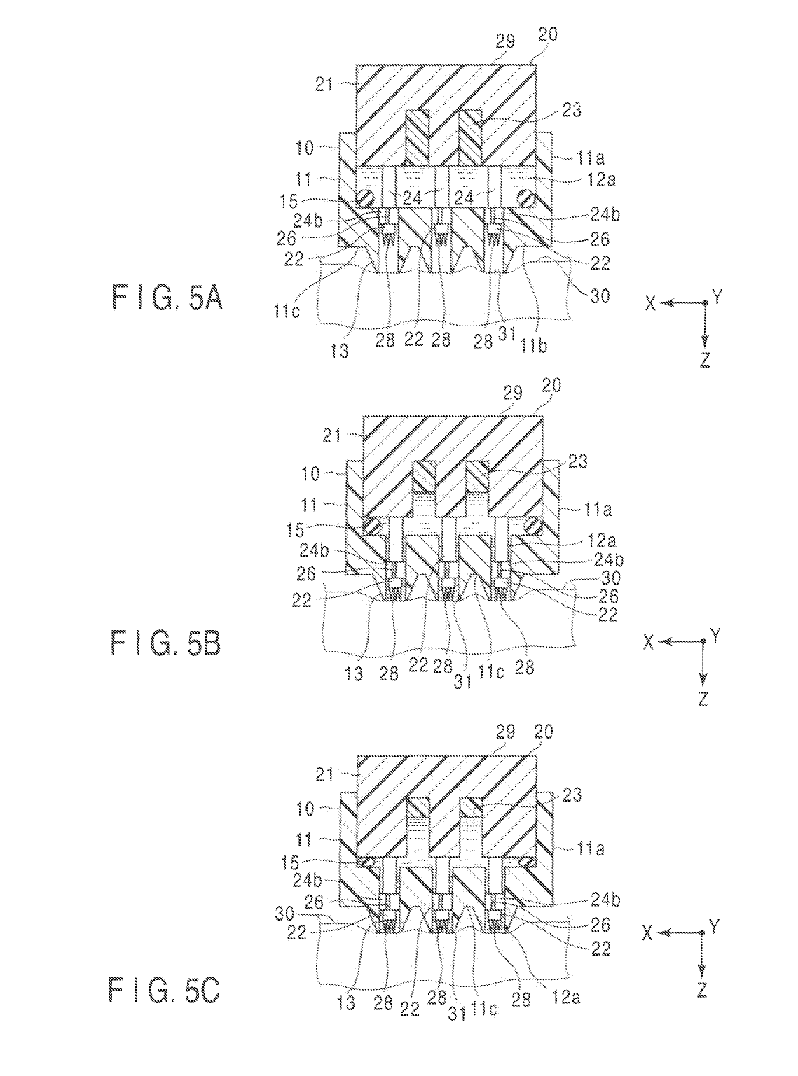

A drug administration method using the transdermal drug administration apparatus 1 of the present embodiment will now be described with reference to FIGS. 5A to 5C, which are diagrams individually showing drug administration processes.

The base section 10 of the transdermal drug administration apparatus 1 is formed to have a diameter of several centimeters such that an operator can hold and operate it with one hand.

The operator holds the transdermal drug administration apparatus 1 and presses the facing surface 11c against the skin 30 so that the surface 11c faces a subject area 31 of the skin 30, as shown in FIG. 5A. If the facing surface 11c is pressed against the skin 30, the protrusions 13 bite into and spread the skin 30, whereupon the surface 11c closely contacts the skin 30. When this is done, the skin 30 in contact with the guide hole 14 in the center of each protrusion 13 is subjected to a tension such that the skin is spread.

Then, the operator depresses the push button 29 in the Z-direction by a finger or the like, thereby pushing the slide section 20 toward the skin 30, as shown in FIG. 5B. Thereupon, the moving part 21 moves in the Z-direction, guided by the peripheral wall portion 11a of the casing 11, and the needle portions 22 move in the Z-direction, guided by the respective inside walls of the guide holes 14. If this is done, the microneedles 28a to 28c on the respective distal ends of needle portions 22 approach the skin 30, and at the same time, the drug 12a is delivered to the respective distal end faces of the protrusions 13 through communication paths that comprise the guide holes 14 and guide grooves 26. When this is done, the elastically-compressed members 23 are compressed, and the drug 12a is also held in the elastic member holes 21a having so far been holding the compressed members 23.

If the push button 29 is further depressed, as shown in FIG. 5C, the microneedles 28a to 28c pierce and perforate the skin 30 and are introduced into a hypodermic tissue. The depth of penetration into the hypodermic tissue is controlled to be about 100 μm, and the barrier layer of the skin 30 is perforated. Thereupon, the O-ring 15 is elastically deformed between the moving part 21 and base section 10.

If the push button 29 is released from the depression, the slide section 20 is retracted from the skin 30 by the elastic restoring force of the O-ring 15, and the needle portions 22 recede from the skin 30 on the facing surface 11c and return to a release position shown in FIG. 5C. Then, this release position is maintained by a locking mechanism (not shown).

In the release position, the elastically-compressed members 23 are pressurized. As the compressed members 23 are restored to their original shape by their restoring force, the liquid drug is gradually pushed out toward the skin 30. Since the skin 30 from which the microneedles 28a to 28c are removed is then perforated, the drug permeates through the resulting perforations and is absorbed by the hypodermic tissue.

The transdermal drug administration apparatus 1 according to the present embodiment can produce the following effects. The base section 10 comprises the protrusions 13 on the facing surface 11c and the guide holes 14 in the protrusions 13, and the slide section 20 comprises the needle portions 22 movable in the guide holes 14. After the protrusions 13 are pressed against the skin 30 to stretch its wrinkles or the like, therefore, the needle portions 22 can be guided along the guide holes 14 and pressed against the skin 30. Thus, the microneedles 28a to 28c can be accurately pushed into the hypodermic tissue by simply pressing the transdermal drug administration apparatus 1 against the skin 30 and sliding it.

Specifically, those areas of the skin 30 to be pierced by the microneedles 28a to 28c are subjected to a tension such that the skin 30 to be treated can be spread and flattened by only pressing the protrusions 13 against the skin. Further, the slide section 20 and needle portions 22 are guided at right angles to the tensioned areas of the skin 30. Thus, the direction of introduction of the microneedles 28a to 28c can be accurately set to be perpendicular to the skin 30. Since the protrusions 13 closely contact the skin 30, moreover, horizontal (X- and Y-direction) displacements can be prevented.

The above-described transdermal drug administration apparatus 1, thus having a simple structure, can be made smaller and lighter, so that it places little burden on a patient and its manufacturing costs can be reduced.

In the transdermal drug administration apparatus 1, the drug 12a is supplied from around the microneedles 28a to 28c through the guide holes 14 and guide grooves 26, so that the influence of pressure loss can be minimized. Specifically, if the communication paths for drug supply are formed in the distal ends of the sharp portions of the microneedles, for example, formable holes are so small that a large pressure loss is caused. If the microneedles are large in number, the drug supply to the skin surface requires use of a large pump. According to the present embodiment, however, the drug 12a is guided through the guide holes 14 and guide grooves 26, so that the pressure loss can be minimized and the necessary application pressure can be reduced. Unlike in the case where the drug is held on needle faces, moreover, there are no restrictions on the amount and type of the drug to be held.

In the embodiment described above, furthermore, the storage recess 12 communicating with the facing surface 11c is pressed by the moving part 21 of the slide section 20 so that the drug 12a is pushed out toward the skin 30. Thus, both operations for perforation and drug supply can be achieved by a single pressing stroke.

Since the protrusions 13 and needle portions 22 are arranged in three positions, they can be stably set to provide a uniform pressure without regard to the curvature of the skin 30 to be treated.

Microarrays can be used for the microneedle units 28 to facilitate mass production. Further, each microneedle unit 28 is provided on the distal end of the shaft 24. Therefore, if the microneedle unit 28 is formed of, for example, silicon, it requires only a small amount of silicon, so that the manufacturing costs can be reduced.

Since the needle portions 22 are surrounded individually by the protrusions 13, the microneedles 28a to 28c cannot be easily subjected directly to horizontal (X- and Y-direction) loads, and hence, can be prevented from being broken. Since the puncture by the microneedles 28a to 28c is performed only at the start, moreover, they pierce the skin 30 for a shorter time than in a case where the drug is supplied with the microneedles penetrating the skin. Thus, the load on the microneedles 28a to 28c can be reduced, so that the microneedles can be prevented from being broken.

Since the indicator 29a on the button is configured to indicate the button depression, accomplishment of the puncture can be visually confirmed.

### Second Embodiment

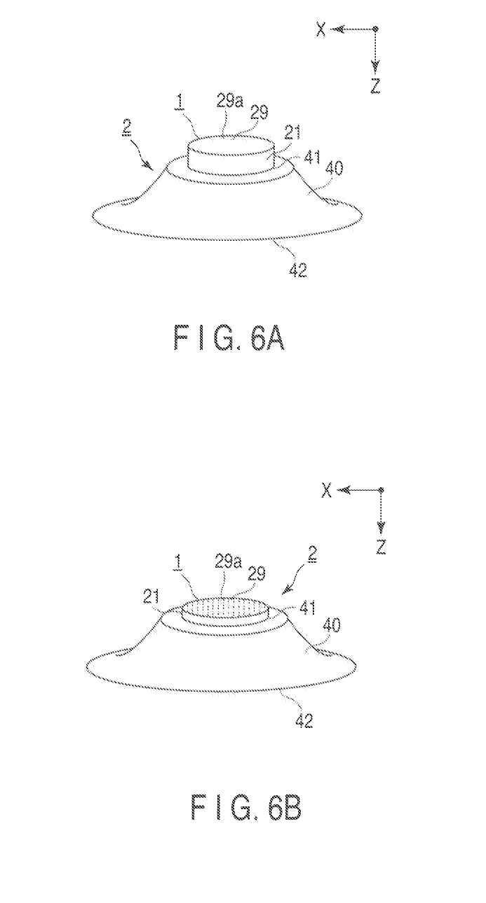

A transdermal drug administration unit 2 according to a second embodiment will now be described with reference to FIGS. 6A and 6B, which are external perspective views of the administration unit 2 using the transdermal drug administration apparatus 1 of the first embodiment, and which show states before and after button depression, respectively. In FIG. 6B, the indicator 29a is hatched in violet or purple. However, the color produced by the indicator 29a is not limited to violet or purple and may be another.

The transdermal drug administration unit 2 comprises the transdermal drug administration apparatus 1 and an attachment seat 40 disposed around the facing surface 11c of the apparatus 1. The attachment seat 40 comprises a circular hole portion 41 in the center and an adhesive layer (attachment portion) 42 below the hole portion 41. The attachment seat 40 is configured so that the moving part 21 of the apparatus 1 is passed through the hole portion 41 and the push button 29 is exposed on the top side. The attachment seat 40 is located at the outer peripheral portion of the casing 11.

In the transdermal drug administration unit 2, the transdermal drug administration apparatus 1 is affixed to the skin 30 by affixing the adhesive layer 42 of the attachment seat 40 around the subject area 31 of the skin 30 with the apparatus 1 pressed against the skin. In this affixed state, as in the case of the first embodiment, perforation and drug supply are achieved by depressing and releasing the push button 29 of the transdermal drug administration apparatus 1. Thereupon, the drug is infiltrated through the perforations in the skin 30.

The same effects as those of the first embodiment can be obtained in the present embodiment. Further, the attachment seat 40 serves for more reliable attachment to the skin 30, improved operability, and prevention of displacement and breakage.

### Third Embodiment

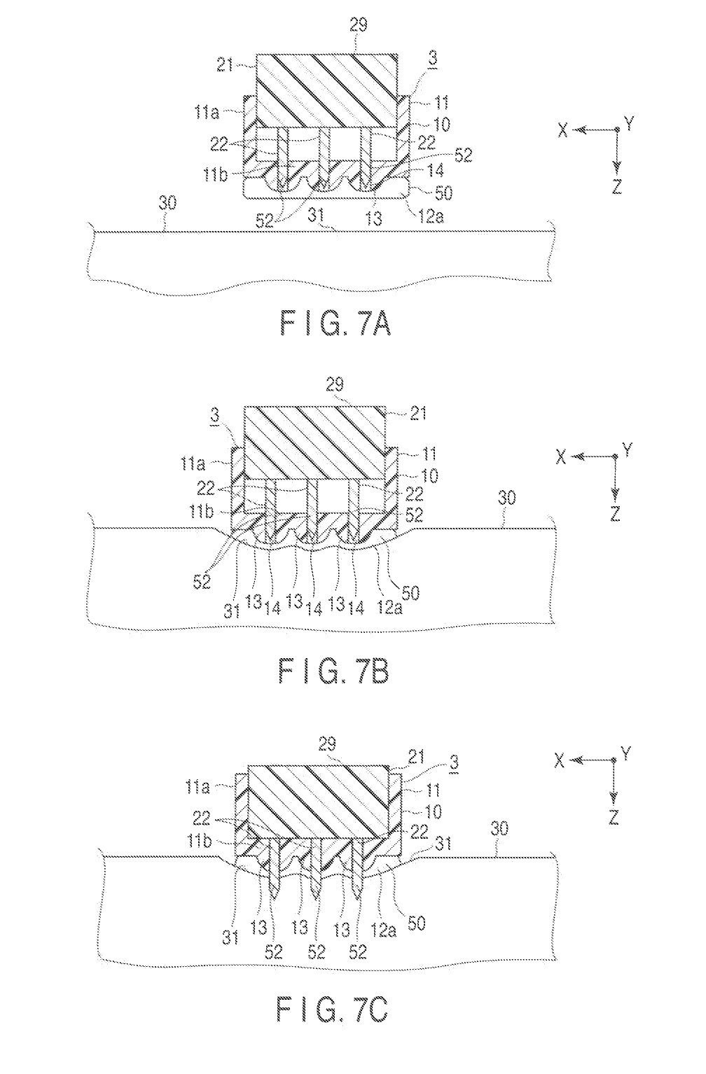

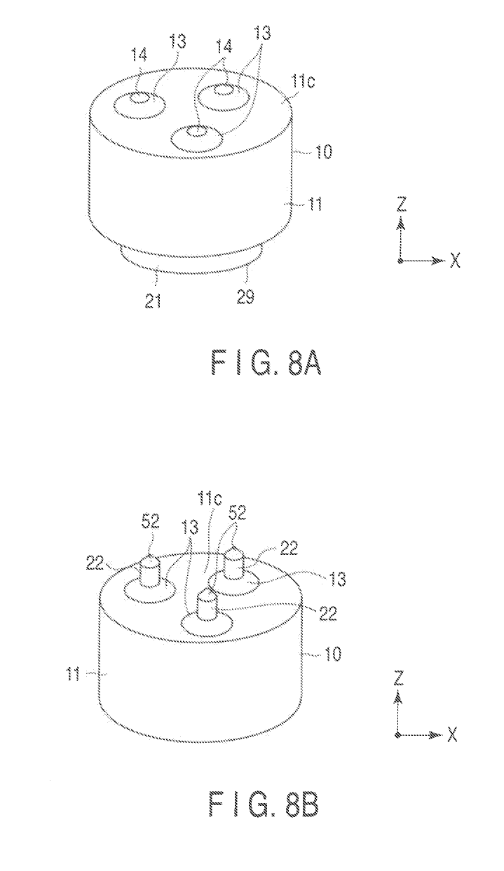

A transdermal drug administration apparatus 3 according to a third embodiment will now be described with reference to FIGS. 7A to 8B. FIGS. 7A to 7C are diagrams showing the internal structure of the administration apparatus 3 of the present embodiment and a drug administration method. FIGS. 8A and 8B, which are perspective views showing an external configuration of the apparatus 3, show a retracted state in which needle portions 22 are retracted and an advanced state in which the needle portions 22 are projected, respectively. The transdermal drug administration apparatus 3 of the present embodiment differs from the transdermal drug administration apparatus 1 of the first embodiment in the shape of the needle portions 22 and in that a drug 12a is held in a gel outside a casing 11. Since other configurations are the same as those of the forgoing embodiments, a repeated description thereof is omitted.

As shown in FIGS. 7A to 8B, the transdermal drug administration apparatus 3 comprises a base section 10 and a slide section 20 movable relative to the base section 10. Protrusions 13 are formed on a facing surface 11c or the lower surface of the base section 10. A gel layer containing the drug 12a is disposed on the facing surface 11c. Specifically, the drug 12a is not held within the base section 10, and a drug gel layer 50 is held on the outer surface of the base section 10. The needle portions 22 are passed individually through guide holes 14 that penetrate a bottom portion 11b of the base section 10.

The three needle portions 22 that extend downward from a moving part 21 of the slide section 20 each comprise a sharp-tip microneedle 52 having a diameter slightly smaller than that of each guide hole 14. The needle portions 22 are contained individually in the guide holes 14 that penetrate the respective centers of the protrusions 13.

If the transdermal drug administration apparatus 3 constructed in this manner is pressed against the skin 30, the protrusions 13 are pushed into the skin 30 to partially spread it after the drug gel layer 50 contacts the skin, as shown in FIG. 7B. Thereupon, the drug gel layer 50 is crushed by the pressure at which the apparatus 3 is pressed against the skin 30 and the drug oozes out.

If the push button 29 is depressed to push in the slide section 20 with the microneedles 52 in the Z-direction, as shown in FIGS. 7C and 8B, the needle portions 22 move along the guide holes so that microneedles 52 on their respective distal ends project from the protrusions 13 and are introduced into a hypodermic tissue. After the microneedles 52 are retracted, the skin 30 has perforations through which the drug 12a contained in the drug gel layer 50 diffuses into the hypodermic tissue.

The same effects as those of the first embodiment can be obtained in the present embodiment. Specifically, those areas of the skin 30 to be pierced by the microneedles 52 are subjected to a tension such that the skin 30 to be treated can be flattened by only pressing the protrusions 13 against the skin. Further, the slide section 20 and needle portions 22 are guided at right angles to the tensioned areas of the skin 30. Thus, the direction of introduction of the microneedles 52 can be accurately set to be perpendicular to the skin 30. Since the protrusions 13 closely contact the skin 30, moreover, horizontal displacements can be prevented. Since the drug 12a can be held in a gel outside the base section 10, furthermore, it can be supplied by means of a simple structure, and the amount of the drug to be held is less restricted.

### Fourth Embodiment

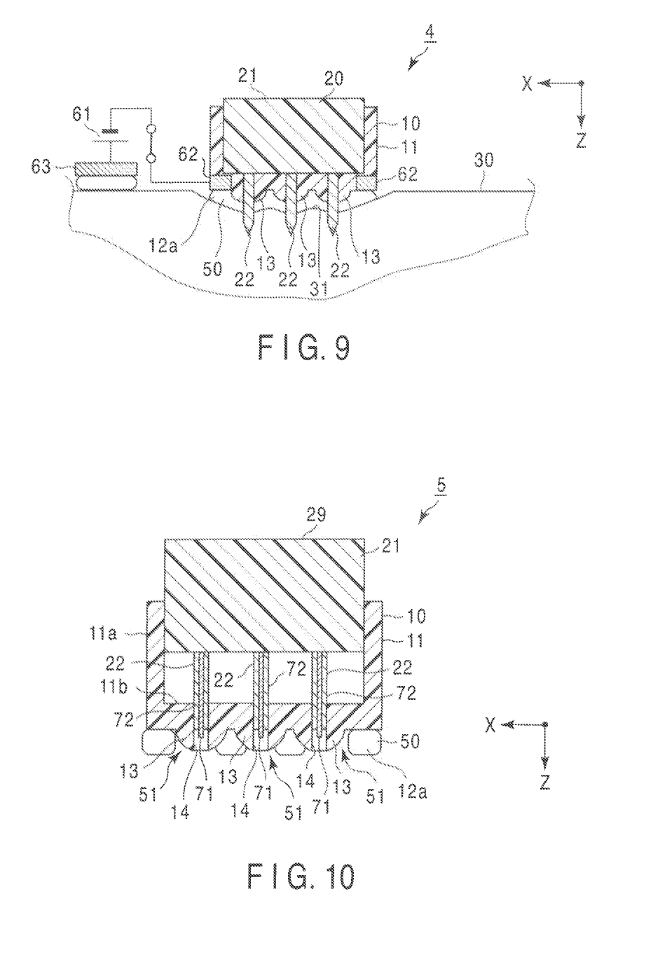

A transdermal drug administration apparatus 4 according to a fourth embodiment will now be described with reference to FIG. 9, which is a diagram showing the internal structure of the administration apparatus 4 of the present embodiment. In the present embodiment, iontophoresis is used in the transdermal drug administration apparatus 3 of the third embodiment. Since other configurations are the same as those of the third embodiment, a repeated description thereof is omitted.

In this transdermal drug administration apparatus 4, a base section 10 is provided with an electrode 62, which contacts a drug gel layer 50. The electrode 62 is connected to a power source 61 and then to a counter electrode 63.

In the transdermal drug administration apparatus 4, the drug is diffused into the hypodermic tissue by the agency of an electric field by bringing the electrode into contact with the skin 30 to apply the electric field thereto.

The transdermal drug administration apparatus 4 of the present embodiment can also produce the same effects as those of the forgoing embodiments. Further, the efficiency of absorption of the drug through the skin 30 can be improved by jointly using iontophoresis.

### Fifth Embodiment

A transdermal drug administration apparatus 5 according to a fifth embodiment will now be described with reference to FIG. 10, which is a diagram showing the internal structure of the administration apparatus 5 of the present embodiment. The apparatus 5 of the present embodiment differs from the apparatus 3 of the third embodiment in that each needle portion 22 comprises a microneedle 71, formed of a titanium wire, and a tube 72 and in the shape of a drug gel layer 50. Since other configurations are the same as those of the third embodiment, a repeated description thereof is omitted.

In the transdermal drug administration apparatus 5 of the present embodiment, each needle portion 22 comprises the tube 72 of polyetheretherketone (PEEK) resin and the microneedle 71, formed of a titanium wire with a diameter of 100 μm or less and configured to be passed through the tube 72.

The drug gel layer 50 comprises holes 51 in regions opposite protrusions 13 such that the layer 50 can interfere with neither the protrusions 13 nor the microneedles 71.

The same effects as those of the forgoing embodiments can be obtained in the present embodiment. Since a metal wire of about 100-μm diameter can be introduced into a hypodermic tissue without having its tip sharpened, so that it can be easily manufactured. The titanium wire is an appropriate material for the microneedle because of its low toxicity to the human body. Products with inside diameters of 100 μm or less are commercially available for the tube 72 of, for example, PEEK resin, and titanium wires of about 100-μm diameters are also on the market. Thus, the needle portions 22 can be easily manufactured by securing these tubes and wires to the slide section 20 by press fitting or the like.

The present embodiments are not limited directly to the embodiments described above, and its constituent elements may be embodied in modified forms without departing from the spirit of the embodiments. For example, the number and specific shapes of protrusions and microneedles, the materials of the members, etc., are not limited to the above-described embodiments. In the second embodiment, the administration unit is formed by providing an attachment seat 40 on the transdermal drug administration apparatus 1 of the first embodiment. However, the attachment seat 40 may also be mounted on any of the other transdermal drug administration apparatuses 3 to 5 to form a transdermal drug administration unit. Further, the drug gel layer 50 of the third embodiment may be provided with holes 51 so as not to interfere with the microneedles, as in the case of the fifth embodiment. In each of the above-described embodiments, moreover, a guide groove may be further formed on each protrusion 13 whereby the oozed drug is guided into the perforations in the skin 30.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A transdermal drug administration apparatus comprising:
a first member comprising a protrusion on a facing surface opposed to a skin and a guide hole in the protrusion; and
a second member comprising a needle portion located in the guide hole and movable in a first direction across a surface of the skin.

2. The transdermal drug administration apparatus according to claim 1, wherein the first member comprises a storage portion configured to accommodate a drug, the guide hole communicates with the interior of the storage portion and the facing surface, the second member comprises a moving part configured to be fitted into the storage portion and the needle portion extending from the moving part toward the skin and is movable relative to the first member in the first direction within the storage portion, and the needle portion projects from the facing surface and perforates the skin and the drug is pushed out through the guide hole by the moving part and delivered toward the facing surface when the second member moves in the first direction toward the skin with the facing surface pressed against the skin.

3. The transdermal drug administration apparatus according to claim 2, further comprising an elastically-compressed member capable of enlargement and reduction on the storage portion side of the moving part, the elastically-compressed member being configured to be compressed and reduced in size between the moving part and the drug as the second member moves and to be enlarged by an elastic restoring force thereof, thereby pushing out the drug in the storage portion toward the facing surface, after a pressure for moving the second member is removed.

4. The transdermal drug administration apparatus according to claim 3, wherein the needle portion comprises a shaft configured to move within the guide hole, one or more needles on the distal end side of the shaft, and a guide groove in a side portion of the shaft, and the drug is delivered to the facing surface through the guide groove and the guide hole.

5. The transdermal drug administration apparatus according to claim 1, wherein the facing surface comprises a drug gel layer containing the drug.

6. The transdermal drug administration apparatus according to claim 1, wherein the second member comprises a pushable portion such that the second member moves toward the facing surface and the needle portion projects from the facing surface and perforates the skin when the pushable portion is depressed, and that the second member moves in a direction to return toward the pushable portion and the needle portion recedes from the facing surface when the pushable portion is released from the depression.

7. The transdermal drug administration apparatus according to claim 6, further comprising an elastic member located between the first and second members and configured to be elastically deformed by a pressure which causes the second member to move toward the facing surface and to push back, by an elastic restoring force thereof, the second member away from the skin when the pressure which causes the second member to move is removed.

8. The transdermal drug administration apparatus according to claim 1, further comprising an electrode provided on the facing surface side and a power source connected to the electrode.

9. The transdermal drug administration apparatus according to claim 6, wherein the pushable portion is formed with an indication portion configured to make an indication when the pushable portion is depressed.

10. A transdermal drug administration unit comprising:
the transdermal drug administration apparatus according to claim 1; and
an attachment seat comprising an attachment portion mounted on the transdermal drug administration apparatus and capable of adhering to the skin in a region around the facing surface.
